(19) United States
(12) Patent Application Publication
(10) Pub. No.: US 2026/0186085 A1
(43) Pub. Date: Jul. 2, 2026

(54) METHOD AND SYSTEM FOR ACQUIRING MAGNETIC FLUX DENSITIES OF A MAGNETIC RESONANCE DEVICE

(71) Applicant: Siemens Healthineers AG, Forchheim (DE)

(72) Inventors: Johann Sukkau, Herzogenaurach (DE); Andrew Dewdney, Neunkirchen am Brand (DE); Andreas Krug, Fürth (DE); Manuela Rösler, Erlangen (DE); Daniel Niederlöhner, Erlangen (DE); Stefan Popescu, Erlangen (DE); Axel vom Endt, Erlangen (DE); Matthias Gebhardt, Erlangen (DE)

(21) Appl. No.: 19/436,701

(22) Filed: Dec. 30, 2025

(30) Foreign Application Priority Data
Dec. 30, 2024 (DE) 10 2024 212 335.8

(51) Int. Cl. G01R 33/24; A61B 5/00; A61B 5/055; G01R 33/385; G01R 33/44; G01R 33/56

(52) U.S. Cl. CPC G01R 33/24; A61B 5/055; A61B 5/70; G01R 33/385; G01R 33/445; G01R 33/5608

(57) ABSTRACT

A method for acquiring magnetic flux densities of a magnetic resonance device within a patient tunnel of the magnetic resonance device includes ascertaining a first spatial direction and/or a second spatial direction in which the magnetic flux density/densities of a B0 magnetic field of a B0 main magnet of the magnetic resonance device within the patient tunnel is or are below a predefined value at at least one position. At least one magnetic field strength sensor is arranged at the at least one position within the patient tunnel. The magnetic field strength sensor is arranged and configured to acquire the magnetic flux density at least in the first spatial direction and/or in the second spatial direction. The magnetic flux density/densities are acquired by the at least one magnetic field strength sensor in the first spatial direction and/or in the second spatial direction.

This application claims the benefit of German Patent Application No. DE 10 2024 212 335.8, filed on Dec. 30, 2024, which is hereby incorporated by reference in its entirety.

### BACKGROUND

The present embodiments relate to acquiring magnetic flux densities of a magnetic resonance device, and to the use of the acquired magnetic flux densities.

Magnetic resonance (MRT, also MRI) devices are image-generating apparatuses that, for the purposes of mapping an object under examination, orient nuclear spins of the object under examination with a strong external magnetic field and excite them to precess around the orientation via an alternating magnetic field. The precession or return of the spins from the excited state into a lower-energy state in turn generates in response an alternating magnetic field that may be received via antennas. Spatial encoding may be impressed onto the signals with the assistance of magnetic gradient fields provided by gradient coils. The encoding subsequently permits assignment of the received signal to a volume element. The received signal may then be evaluated, and a three-dimensional image representing the object under examination may be provided. This procedure is known as image reconstruction. The magnetic gradient field as actually provided during an examination is of particular significance (e.g., for image reconstruction). In particular, very stringent requirements apply to the magnetic fields used for generating magnetic resonance images.

It has been found in this connection that there is a need to provide a method and a system with which it is possible to acquire magnetic flux densities of a magnetic resonance device (e.g., magnetic flux densities of a gradient coil of a magnetic resonance device) and changes in magnetic flux densities caused by activating a gradient coil.

U.S. Pat. No. 10,641,858 B2 describes an MRI system in which magnetic field sensors are used to measure transverse components of the magnetic field along the x, y, and z axes. Signals from these sensors are used to determine gradients of the magnetic gradient field along the longitudinal axis.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a method and a system with which magnetic flux densities of a magnetic resonance device, such as of a gradient coil of a magnetic resonance device, and/or their changes may be acquired is provided.

The present embodiments include a method for acquiring magnetic flux densities of a magnetic resonance device within a patient tunnel of the magnetic resonance device.

The method includes: ascertaining a first spatial direction and/or a second spatial direction in which the magnetic flux density/densities of a B0 magnetic field of a B0 main magnet of the magnetic resonance device within the patient tunnel is or are below a predefined value at at least one position; arranging at least one magnetic field strength sensor at the at least one position within the patient tunnel, where the magnetic field strength sensor is arranged and set up in such a way as to acquire the magnetic flux density at least in the first spatial direction and/or in the second spatial direction; and acquiring the magnetic flux density/densities using the at least one magnetic field strength sensor in the first spatial direction and/or in the second spatial direction.

One aspect of the present embodiments provides a method for acquiring magnetic flux densities of a gradient coil of a magnetic resonance device within a patient tunnel of the magnetic resonance device. The method includes ascertaining at least one position within the patient tunnel at which the magnetic flux densities of a B0 magnetic field of a B0 main magnet of the magnetic resonance device are below a predefined value in a first spatial direction and in a second spatial direction; arranging at least one magnetic field strength sensor at the at least one position, wherein the magnetic field strength sensor is arranged and set up in such a way as to acquire the magnetic flux density at least in the first spatial direction and in the second spatial direction; and acquiring the magnetic flux densities by means of the at least one magnetic field strength sensor in the first spatial direction and in the second spatial direction.

One aspect of the present embodiments provides a method for acquiring magnetic flux densities of a magnetic resonance device within a patient tunnel of the magnetic resonance device. The method includes: ascertaining a first spatial direction in which the magnetic flux density/densities of a B0 magnetic field of a B0 main magnet of the magnetic resonance device within the patient tunnel is or are below a predefined value at at least one position; arranging at least one magnetic field strength sensor at the at least one position within the patient tunnel, where the magnetic field strength sensor is arranged and set up in such a way as to acquire the magnetic flux density at least in the first spatial direction; and acquiring the magnetic flux density/densities using the at least one magnetic field strength sensor in the first spatial direction.

Elevated magnetic flux densities are used in magnetic resonance devices. For example, magnetic resonance devices are known in which a B0 main magnet has a magnetic flux density of 3 T. Acquiring or measuring magnetic flux densities of the gradient coil within a patient tunnel of a magnetic resonance device via a magnetic field strength sensor (e.g., via a three-dimensional Hall sensor) is therefore problematic to the extent that such magnetic field strength sensors typically exhibit a relative measurement error in the percentage range. Magnetic field strength sensors with a measurement range of 3 T and a relative error of, for example, 1% exhibit an absolute error of 30 mT. Such magnetic field strength sensors thus cannot be used to acquire a magnetic flux density or a change in magnetic flux density of the gradient coils with sufficiently elevated accuracy in order to acquire, for example, fluctuations or imperfections in the gradient fields in the microtesla range. If magnetic field strength sensors of appropriately elevated accuracy are used (e.g., with a linear measurement range in the microtesla range), such a magnetic field strength sensor passes into its saturation range at a magnetic flux density of 3 T, such that reliable measured values may again no longer be provided. The use of the term “magnetic flux density” may be interpreted broadly for the purposes of the present embodiments. For example, the magnetic flux density may also denote a magnetic field strength. Inasmuch as a magnetic flux density is measured, a magnetic field strength may also be understood as being measured.

The present embodiments are based on the insight that a B0 magnetic field of a B0 main magnet of a magnetic resonance device in the homogeneity range has an elevated magnetic field strength component in just one spatial direction (e.g., in the spatial direction parallel to the axis of symmetry of the B0 magnetic field of the B0 main magnet).

An X-Y-Z coordinate system is conventionally used as the basis for spatial encoding and for a more detailed description of spatial directions and the like. The Z coordinate axis is conventionally, and here too, defined as an axis of symmetry of the B0 main magnet of the magnetic resonance device through a patient tunnel of the B0 main magnet in the preferential direction of the B0 field. In the conventional setup of a magnetic resonance device, the Z coordinate axis is oriented horizontally and extends centrally through the opening of the windings of the B0 main magnet. The capture object is conventionally pushed into the patient tunnel parallel to the Z coordinate axis on a mobile patient table. Together with the Z coordinate axis, an X coordinate axis and a Y coordinate axis enclose a space. The coordinate axes may be provided orthogonally to one another, the X coordinate axis may be oriented horizontally, and the Y coordinate axis may be oriented vertically.

Based on this X-Y-Z coordinate system, in the homogeneity range of a B0 main magnet, a relevant field strength component of the B0 magnetic field is thus only present in the Z coordinate axis. The component is also denoted Z field strength component (B0.z). The field strength components in the X coordinate axis and the Y coordinate axis are correspondingly conventionally denoted X field strength component (B0.x) and Y field strength component (B0.y).

The present embodiments are also based on the insight that the B0 magnetic field of the B0 main magnet has a plurality of positions at which both the X field strength component (B0.x) and the Y field strength component (B0.y) are substantially zero (e.g., the magnetic flux densities in these two spatial directions are substantially 0). These positions at which the magnetic flux densities are below a predefined value, and, for example, amount substantially to 0, are ascertained in order to place the magnetic field strength sensors. In a configuration of the present embodiments, the (measurement) axes of the magnetic field strength sensor are oriented collinearly to X field strength component (B0.x) and Y field strength component (B0.y). If, for example, a three-dimensional magnetic field strength sensor (e.g., a three-dimensional Hall sensor) is used and the measurement axes are arranged collinearly to the axes of the X-Y-Z coordinate system, the magnetic flux density or also small changes in magnetic flux density in the X and Y directions may be acquired, irrespective of the fact that an elevated magnetic flux density is present in the Z direction and a magnetic field strength sensor for the Z field strength component (B0.z) is reaching saturation. It is therefore possible to use a magnetic field strength sensor with high accuracy and to acquire the magnetic flux densities in the X and Y directions with a correspondingly high accuracy.

In other words, at least one aspect of the present embodiments provides initially ascertaining positions within the patient tunnel at which the X field strength component (B0.x) and/or the Y field strength component (B0.y) of the B0 magnetic field of the B0 main magnet is/are below a specified value (e.g., amount(s) substantially to 0), and arranging one or more magnetic field strength sensors at these positions in order to acquire/measure the X field strength component (B0.x) and Y field strength component (B0.y) there. These positions may be ascertained in different ways (e.g., the magnetic field may be measured or also be provided by calculation or simulation). These positions may, for example, be calculated with the assistance of the geometry of the B0 main magnet. A map of the B0 magnetic field from which suitable positions may be derived may also be available for a magnetic resonance device.

The procedure of the present embodiments may be used to acquire the magnetic flux densities of the gradient fields and directly measure their changes. The acquired magnetic flux densities may then be used for various applications. For example, it is possible for the gradient fields or the change in the gradient fields to be continuously acquired and monitored. The acquired magnetic flux densities may be used, for example, for the purposes of image reconstruction (e.g., in the event of undesired eddy current effects). It is also possible to monitor the magnetic flux densities and perform an emergency shutdown should magnetic flux densities fall outside a predefined range. It is consequently also possible to carry out a nominal/actual comparison of the magnetic flux densities (e.g., in order to be able to carry out quality and/or operating status evaluations with regard to the gradient coil). Finally, the Z field strength component may also be ascertained based on the X and Y field strength components.

The term “patient tunnel” may be interpreted broadly here. For example, the term “patient tunnel” is intended to be the interior of the “tube” or “bore” typical of most magnetic resonance devices, but the “patient tunnel” may also be the sensitive region suitable for imaging of a magnetic resonance device of any desired configuration.

As explained, the axes of the magnetic field strength sensor may be oriented collinearly at least to the two spatial directions (e.g., X field strength component (B0.x) and Y field strength component (B0.y)) of the B0 magnetic field or to at least one of the two spatial directions, the magnetic flux densities of which are acquired. The present disclosure is, however, not limited thereto. Depending on the configuration, the axes of the magnetic field strength sensor may also be arranged non-collinearly or in an angular range to the two spatial directions.

The predefined value of the magnetic flux densities may be between −0.02 T and 0.02 T, between −0.01 T and 0.01 T, or between −5 μT and 5 μT.

As already explained, according to one variant, the magnetic field strength sensor is a three-dimensional magnetic field strength sensor and is configured to acquire a field strength of three components of the B0 magnetic field in three spatial directions. The axes of the magnetic field strength sensor may again be oriented collinearly to the three spatial directions of the B0 magnetic field. The present disclosure is, however, not limited to the use of a three-dimensional magnetic field strength sensor. Additionally and/or alternatively, two-dimensional magnetic field strength sensors that are again oriented collinearly to the two spatial directions (X field strength component (B0.x) and Y field strength component (B0.y)) of the B0 magnetic field may also be used.

The magnetic field strength sensor may be a Hall sensor, an anisotropic magnetoresistive sensor, a magnetic tunnel resistance sensor, or a fluxgate magnetometer. Various magnetic field strength sensors may also be used in one embodiment. The at least one magnetic field strength sensor may have a linear measurement range between 0.01 T and −0.01 T.

Magnetic field strength sensors may be arranged at different positions within the patient tunnel at which the magnetic flux densities of the B0 magnetic field of the B0 main magnet of the magnetic resonance device are below the predefined value in the first spatial direction and in the second spatial direction. The magnetic field strength sensors are in each case arranged and configured in such a way as to acquire the magnetic flux density at least in the first spatial direction and in the second spatial direction. One or more “sensor arrays” of a plurality of magnetic field sensors, which may be placed not only adjacent one another but also above one another, may be used.

The method may also include the act of ascertaining a magnetic flux density in the third spatial direction based on the acquired magnetic flux densities in the first spatial direction and in the second spatial direction. In the configuration, the X and Y field strength components are acquired as described above. It is then possible to calculate or derive the Z field strength component based on the “source freedom” of the magnetic flux density since there are only closed field lines in the magnetic field. Applying Maxwell's second equation, which is also known as “Gauss' law for magnetic fields”, gives rise to the following:

$$∇ \cdot B = 0$$

which states that the magnetic field is source-free. The divergence of the vector field is:

$$B = ( {B}_{x} , {B}_{y} , {B}_{z} )$$

and is defined as

$$\frac{\partial {B}_{x}}{\partial x} + \frac{\partial {B}_{y}}{\partial y} + \frac{\partial {B}_{z}}{\partial z} = 0 ,$$

and, solved according to

$$\frac{\partial {B}_{z}}{\partial z} ,$$

gives rise to:

$$- ( \frac{\partial {B}_{x}}{\partial x} + \frac{\partial {B}_{y}}{\partial y} ) = \frac{\partial {B}_{z}}{\partial z}$$

Placing a plurality of magnetic field strength sensors therefore makes it possible not only to directly measure the magnetic flux densities (e.g., the local X and Y field strength components), but also to determine the corresponding derivatives in both spatial directions in order to be able to calculate the magnetic flux density in the Z direction.

According to one embodiment, the at least one magnetic field strength sensor includes a plurality of magnetic field strength sensors that are arranged at a plurality of positions (e.g., at least four different positions, at least ten positions, or at least 20 positions) within the patient tunnel. The magnetic field strength sensor is arranged and configured in such a way as to acquire the magnetic flux density in the first spatial direction. The magnetic flux densities are acquired in the first spatial direction by the magnetic field strength sensors. Based on the acquired magnetic flux densities in the first spatial direction, the magnetic flux densities and/or the local derivatives of the magnetic flux densities in the second spatial direction and/or in a third spatial direction are ascertained (e.g., ascertained by calculation). For example, a location-dependent magnetic flux density in a defined volume is ascertained in at least two (e.g., in three) spatial directions. The magnetic field strength sensor may, for example, be configured and arranged to acquire the magnetic flux density only in the first spatial direction. For example, all the magnetic field strength sensors may be configured and arranged to acquire the magnetic flux density only in the first spatial direction. In this context, the first spatial direction may, for example, be a spatial direction that is perpendicular to the spatial direction of the B0 field and/or the (longitudinal) axis of symmetry of the B0 magnet. For example, the first spatial direction may be the X direction or the Y direction of the system or a linear combination of the X direction and the Y direction. It has been established for the purposes of the present embodiments that acquiring the magnetic flux density in just one spatial direction may in itself be sufficient also to ascertain the magnetic flux density in further spatial directions. For example, provision may be made to ascertain a profile of the magnetic flux densities in the patient tunnel and/or in a defined volume (e.g., a defined volume in the patient tunnel) for all three spatial directions. The magnetic field strength sensors may, for example, be placed in the peripheral zone of a or the defined volume. For example, provision may be made for signals from the magnetic field strength sensors in each case to be acquired substantially simultaneously and for the ascertained derivatives and/or magnetic flux densities in each case to be ascertained for these simultaneously acquired signals.

Measurement of the magnetic flux densities in the first spatial direction may be repeated over time (e.g., repeated a number of times). Measurement may be repeated, for example, at identical time intervals or virtually continuously. A magnetic field strength sensor that ascertains magnetic flux density in only the first spatial direction may be particularly inexpensive and simple to produce. The first spatial direction is, for example, oriented orthogonally to the second spatial direction and/or the third spatial direction.

According to one embodiment, the magnetic flux densities in the second spatial direction and/or in a third spatial direction are ascertained based on calculations using spatial harmonic functions (e.g., regular spatial harmonic functions). It has been found that, due to the geometric circumstances, spatial harmonic functions are particularly well suited to carrying out the calculations for the purposes of the present embodiments. For example, this provides that just one spatial direction is needed to directly ascertain therefrom the local derivatives of the two further spatial directions. Provision may be made for a first field gradient model in the patient tunnel and/or in a defined volume in the patient tunnel to be ascertained based on the ascertained flux densities. A field gradient model may also be denoted an incomplete field model. The first field gradient model may be obtained, for example, by fitting a (e.g., complete) first field model (e.g., a full field model) to the measured values from the magnetic field strength sensors using spatial harmonic functions. Appropriate coefficients of the first field model may be adapted to the measured magnetic flux densities. A second field gradient model along the second spatial direction or along the third spatial direction may be calculated based on the first field gradient model of the first spatial direction. A third field gradient model along the remaining spatial direction in each case (e.g., along the third spatial direction) may be calculated based on the first field gradient model of the first spatial direction or based on the already calculated second field gradient model. A first-order model coefficient for the field model may also be calculated (e.g., a first-order model coefficient for the second field model and/or a first-order model coefficient for the third field model). Additional measurements (e.g., measurements of magnetic flux density) or estimates made on the assumption that magnetic fields decay over greater distances may, for example, be used for calculating the model coefficients.

According to one embodiment, at least two field probes that are configured to acquire a local absolute value of a magnetic field strength are also arranged in the patient tunnel, where an absolute value of the magnetic field strength is in each case acquired by the at least two field probes. The magnetic flux densities are ascertained in the second spatial direction and/or in a third spatial direction both based on the acquired magnetic flux densities in the first spatial direction and based on the absolute values of magnetic field strength ascertained with the at least two field probes. Using (at least) two field probes may be a particularly simple possible way not only of ascertaining the local derivative of the magnetic flux densities in the two further spatial directions, but also of ascertaining the actual flux densities. Provision may be made for signals from the field probes to be acquired substantially simultaneously with the magnetic field strength sensors and for the ascertained magnetic flux densities in each case to be ascertained for these simultaneously acquired signals. A first-order model coefficient for the above-stated second field model and/or the third field model (e.g., a first-order model coefficient for the second field model and/or a first-order model coefficient for the third field model) may in each case be calculated using the local measurements from the field probes.

The location-dependent magnetic flux density within a or the above-stated defined volume may generally be calculated based on the first field model, the second field model, and/or the third field model. The location-dependent magnetic flux density may, for example, be calculated for different points in time (e.g., corresponding to the points in time at which the magnetic flux density is measured in the first spatial direction).

The mathematical or physical principles and an example of the calculation of the magnetic flux densities in the second spatial direction or the third spatial direction or the location-dependent magnetic flux density in a plurality of spatial directions starting from the measured flux density in the first spatial direction are stated below.

A spherical harmonic function is defined as the solution of the spherical harmonic differential equation that is given as the angular fraction of the Laplace equation in spherical coordinates on the spherical surface (r=r₀=constant). Since the spherical harmonic functions form a complete set of orthogonal functions with an orthonormal basis on the spherical surface, any function defined on the spherical surface may be expressed as a weighted sum of these spherical harmonic functions. A spatial harmonic function, also denoted a solid harmonic function, is defined as a well-defined solution of the Laplace equation in the interior of a sphere (r>r₀). A distinction may be drawn between regular spatial harmonic functions that are well-defined at the origin, and irregular spatial harmonic functions that have a singularity at the origin. Regular spatial harmonic functions in Cartesian coordinates are the starting point in the following example. Since the spatial harmonic functions form a complete set of orthogonal functions with an orthonormal basis in three-dimensional space, any function defined in the interior of a sphere may be expressed as a weighted sum of these spatial harmonic functions.

In general, a three-dimensional scalar function (r) or a three-dimensional vector function F(r), for which Δ·f(r)=0 or Δ·F(r)=0 respectively apply (e.g., Laplace equations with the Laplace operator Δ), may be denoted a harmonic function or harmonic field vector. Spatial harmonic functions (also known as solid harmonic functions) are orthogonal solutions of these Laplace equations. In principle, any harmonic function or any harmonic field may be represented by an appropriately calibrated weighted sum of such spatial harmonic functions. Within spatial regions that are free of electrical currents, the vector field of magnetic flux density B is a harmonic field vector, and its Cartesian components Bₓ, B_{y}, and B_{z} are harmonic functions. According to the Maxwell equations, ∇×B and V. B are in each case zero (e.g., B is an eddy-free and source-free field).

Three-dimensional field distributions may be determined by fitting N scalar coefficients Cᵢ (i=1 . . . N) of a linear decomposition of a target field into a weighted sum of N spatial harmonic (SH) functions. This may be represented as follows (e.g., for the three spatial directions x, y, z):

$${B}_{x} ( r , t ) = {\sum }_{i = 1}^{N} C {X}_{i} ( t ) \cdot {SH}_{i} ( r )$$

$${B}_{y} ( r , t ) = {\sum }_{i = 1}^{N} {CY}_{i} ( t ) \cdot {SH}_{i} ( r )$$

$${B}_{z} ( r , t ) = {\sum }_{i = 1}^{N} C {Z}_{i} ( t ) \cdot {SH}_{i} ( r )$$

The index i denotes a spatial harmonic function of I^{th} degree and of order

$$m = - l , 0 , l$$

Table 1 sets out the first 16 regular spatial harmonic functions up to the third degree. These terms may serve as examples for field models. Completeness of these field models provides that this development for a sufficient number of terms N may converge to an exact result. The set of scalar coefficients Cᵢ enables the calculation of the Cartesian components of the magnetic field or of the magnetic flux density at any desired location r (x,y,z) in a volume. Such a set of coefficients can be denoted a full field model (FFM) or complete field model.

TABLE 1

| De- | In- | Spatial harmonic | First partial derivatives | | |
|---|---|---|---|---|---|
| gree 1 | dex i | functions SHᵢ(x, y, z) | $\frac{\partial {SH}_{i}}{\partial x}$ | $\frac{\partial {SH}_{i}}{\partial y}$ | $\frac{\partial {SH}_{i}}{\partial z}$ |
| 0 | 1 | 1 | 0 | 0 | 0 |
| 1 | 2 | x | 1 | 0 | 0 |
| | 3 | y | 0 | 1 | 0 |
| | 4 | z | 0 | 0 | 1 |
| 2 | 5 | xy | y | x | 0 |
| | 6 | zy | 0 | z | y |
| | 7 | 2z²−x²−y² | −2x | −2y | 4z |
| | 8 | xz | z | 0 | x |
| | 9 | x²−y² | 2x | −2y | 0 |
| 3 | 10 | 3x²y−y³ | 6xy | 3x²−3y² | 0 |
| | 11 | xyz | yz | xz | xy |
| | 12 | 4yz²−x²y−y³ | −2xy | 4z²−x²−3y² | 8yz |
| | 13 | 2z³−3x²z−3y²z | −6xz | −6yz | 6z²−3x²−3y² |
| | 14 | 4xz²−x³−xy² | 4z²−3x²−y² | −2xy | 8xz |
| | 15 | x²z−y²z | 2xz | −2yz | x²−y² |
| | 16 | x³−3xy² | 3x²−3y² | −6xy | 0 |

The field gradient models may be derived based on the field models. The first three partial derivatives of the Cartesian components may be used for this purpose. For example, the field gradient model for Bₓ has three parts that may be completely described by N−1 model coefficients CXᵢ(t), i=2 . . . N:

$$\frac{\partial {B}_{x}}{\partial x} ( r , t ) = {\sum }_{i = 2}^{N} C {X}_{i} ( t ) \cdot \frac{\partial {SH}_{i}}{\partial x} ( r )$$

$$\frac{\partial {B}_{x}}{\partial y} ( r , t ) = {\sum }_{i = 2}^{N} C {X}_{i} ( t ) \cdot \frac{\partial {SH}_{i}}{\partial y} ( r )$$

$$\frac{\partial {B}_{x}}{\partial z} ( r , t ) = {\sum }_{i = 2}^{N} C {X}_{i} ( t ) \cdot \frac{\partial {SH}_{i}}{\partial z} ( r )$$

$$Because$$

$$\frac{\partial {SH}_{1}}{\partial x} ( r ) = \frac{\partial {SH}_{1}}{\partial y} ( r ) = \frac{\partial {SH}_{1}}{\partial z} ( r ) = 0$$

applies (see Table 1), a field gradient model in this example effectively has only N−1 terms that are determined by the corresponding (final)N−1 model coefficients of the original field model (e.g., FFM). The reduced number of N−1 model coefficients (e.g., CX₂(t) . . . CX_{N}(t)) may be denoted an incomplete field model (IFM) or also a field gradient model. Corresponding expressions may similarly also be drawn up for the Y gradient and the Z gradient of the Cartesian components of the magnetic fields or the magnetic flux densities B_{y} and B_{z}. These, for example, substantially differ in terms of the model coefficients CYᵢ(t) for B_{y} and CZᵢ(t) for B_{z}. A total of nine mathematical expressions are thus obtained with which all nine Cartesian field gradients may be calculated at any desired location r(x,y,z) in the defined volume.

For example, it is possible to use the same set of model coefficients both for the respective field model and for the respective field gradient model of the respective spatial direction or of the respective Cartesian components Bₓ, B_{y} or B_{z} of the magnetic field or the magnetic flux density.

Due to the absence of eddies (∇×\vec{B}=0) and the source freedom (∇·\vec{B}=0) of the magnetic vector field and magnetic flux density,

$$∇ \times B = \vert \begin{matrix}i & j & k \\ \frac{\partial }{\partial x} & \frac{\partial }{\partial y} & \frac{\partial }{\partial y} \\ {B}_{x} & {B}_{y} & {B}_{z}\end{matrix} \vert = 0$$

yields the equations

$$\frac{\partial {B}_{x}}{\partial z} = \frac{\partial {B}_{z}}{\partial x} , \frac{\partial {B}_{y}}{\partial x} = \frac{\partial {B}_{x}}{\partial y} , \frac{\partial {B}_{z}}{\partial y} = \frac{\partial {B}_{y}}{\partial z}$$

$$and$$

$$∇ \cdot \vec{B} = \frac{\partial {B}_{x}}{\partial x} , \frac{\partial {B}_{y}}{\partial y} = \frac{\partial {B}_{z}}{\partial z} = 0$$

$$yields$$

$$\frac{\partial {B}_{Z}}{\partial z} = - ( \frac{\partial {B}_{x}}{\partial x} + \frac{\partial {B}_{y}}{\partial y} )$$

The field gradients of the Cartesian components are therefore not completely mutually independent but may in each case be calculated starting from one another. For example, all the field gradients B_{z} may be calculated from known field gradients Bₓ and/or B_{y}.

The N coefficients CXᵢ of the complete field model (e.g., full field model) may be calculated using the field values at M different measurement locations, where M≥N. A larger number of measurement locations M may enable better measurement accuracy. Ten to 50, or 20 to 30, measurement locations have proven to be particularly advantageous. The number of measurement locations corresponds, for example, to the number of magnetic field strength sensors. By formulating the equation of a field model for all known field values, it is possible to draw up a linear system with M>N equations with N unknowns, which are the model coefficients CXᵢ:

$${\sum }_{i = 1}^{N} C {X}_{i} ( {t}_{j} ) \cdot {SH}_{i} ( {r}_{k} ) = {B}_{x} ( {r}_{k} , {t}_{j} ) , k = 1 … M$$

This linear system may be solved using known arithmetic or with the assistance of numerical algorithms in order to find the N coefficients of the complete field model (e.g., in this example, for Bₓ). Using the complete field model, it is then possible to calculate the field values or the values of the magnetic flux density Bₓ, (rₖ, tⱼ) at any desired point rₖ(xₖ,yₖ,zₖ) (e.g., also away from the original measurement locations).

Once the model coefficients of the first field model have been determined, the field gradient model for Bₓ may be ascertained using the first partial derivatives of the complete field model:

$$\frac{\partial {B}_{x}}{\partial x} ( r , {t}_{j} ) = {\sum }_{i = 2}^{N} {CX}_{i} ( {t}_{j} ) \cdot \frac{\partial {SH}_{i}}{\partial x} ( r )$$

$$\frac{\partial {B}_{x}}{\partial y} ( r , {t}_{j} ) = {\sum }_{i = 2}^{N} {CX}_{i} ( {t}_{j} ) \cdot \frac{\partial {SH}_{i}}{\partial y} ( r )$$

$$\frac{\partial {B}_{x}}{\partial z} ( r , {t}_{j} ) = {\sum }_{i = 2}^{N} {CX}_{i} ( {t}_{j} ) \cdot \frac{\partial {SH}_{i}}{\partial z} ( r )$$

Using these equations, all three magnetic field gradients for Bₓ may be calculated at any point r within the defined volume. It has been recognized for the purposes of the present embodiments that the same model coefficients may be used for the complete field models and the field gradient models. These equations also apply correspondingly to the Cartesian components B_{y} and B_{z} of the other spatial directions by simple replacement of the model coefficients CX; in each case with CY; for B_{y} and CZᵢ for B_{z}, respectively.

Using the above equations for the first field gradient model and the underlying relationship

$$\frac{\partial {B}_{y}}{\partial x} = \frac{\partial {B}_{x}}{\partial y}$$

the values of the second field gradient may be calculated at (at least) M≥N−1 points rₖ with k=1 . . . M and using

$$\frac{\partial {B}_{y}}{\partial x} ( {r}_{k} , {t}_{j} ) = \frac{\partial {B}_{X}}{\partial y} ( {r}_{k} , {t}_{j} ) = {\sum }_{i = 2}^{N} {CX}_{i} ( {t}_{j} ) \cdot \frac{\partial {SH}_{i}}{\partial y} ( {r}_{k} ) .$$

The X gradient for By may thus be calculated at any point r within the defined volume. A linear system with M equations and N−1 unknowns CYᵢ, i=2 . . . N is generated here:

$${\sum }_{i = 2}^{N} {CY}_{i} ( {t}_{j} ) \cdot \frac{\partial {SH}_{i}}{\partial y} ( {r}_{k} ) = \frac{\partial {B}_{x}}{\partial y} ( {r}_{k} , {t}_{j} )$$

This linear equation system may be solved using arithmetic or using numerical algorithms in order to determine the model coefficients of CYᵢ, i=2 . . . N of the second field gradient model.

Using the above equations for the first field gradient model and the underlying relationship

$$\frac{\partial {B}_{z}}{\partial x} = \frac{\partial {B}_{x}}{\partial z}$$

the values of the third field gradient may be calculated at (at least) M≥N−1 points rₖ with k=1 . . . M and using

$$\frac{\partial {B}_{z}}{\partial x} ( {r}_{k} , {t}_{j} ) = \frac{\partial {B}_{x}}{\partial z} ( {r}_{k} , {t}_{j} ) = {\sum }_{i = 2}^{N} {CX}_{i} ( {t}_{j} ) \cdot \frac{\partial {SH}_{i}}{\partial z} ( {r}_{k} ) .$$

The X gradient for B_{z} may thus be calculated at any point r within the defined volume. A linear system with M equations and N−1 unknowns CZᵢ, i=2 . . . N is generated here:

$${\sum }_{i = 2}^{N} {CZ}_{i} ( {t}_{j} ) \cdot \frac{\partial {SH}_{i}}{\partial z} ( {r}_{k} ) = \frac{\partial {B}_{x}}{\partial z} ( {r}_{k} , {t}_{j} )$$

This linear equation system may be solved using arithmetic or using numerical algorithms in order to determine the model coefficients of CZᵢ, i=2 . . . N of the second field gradient model.

The calculation method shown here should merely be understood as one possible example. Other variants for determining the field gradient models are also possible. In particular, there are many possible ways in which the field gradient models may be determined using the spatial harmonic functions. For example, the third field gradient model may first be calculated from the first gradient model, and then the second field gradient model may be determined from the first field gradient model or the third field gradient model.

It is finally shown by way of example here how the first-order model coefficients may be determined with the assistance of the measured values from the field probes. The field probes each provide an absolute value of the magnetic field or the magnetic flux density for two different locations rₐ and r_{b}. These absolute values may be expressed as follows using the above field models:

$${B}^{3} ( r , {t}_{j} ) = {B}_{x}^{2} ( r , {t}_{j} ) + {B}_{y}^{2} ( r , {t}_{j} ) + {B}_{x}^{2} ( r , {t}_{j} ) = {B}_{x}^{2} ( r , {t}_{j} ) + {( {CY}_{1} ( {t}_{j} ) + {\sum }_{i = 2}^{N} {CY}_{i} ( {t}_{j} ) \cdot {SH}_{i} ( r ) )}^{2} + {( {CZ}_{1} ( {t}_{j} ) + {\sum }_{i = 2}^{N} {CZ}_{i} ( {t}_{j} ) \cdot {SH}_{i} ( r ) )}^{2}$$

Only two unknowns remain in the equation, the model coefficients of the second field model CY₁ (e.g., for the By component) and of the third field model CZ₁ (e.g., for the B_{z} component). By formulating this equation for the two positions of the field probes (e.g., r=rₐ and r=r_{b}), a system including two quadratic linear equations with two unknowns that may be solved using arithmetic or numerical algorithms in order to find the missing first-order model coefficients CY₁ and CZ₁ is obtained.

All the model coefficients of the three complete field models are thus known. Using the equations of the field models, it is therefore possible to determine the Cartesian components Bₓ, B_{y}, B_{z} at any desired location r(x, y, z) within the defined volume. The local magnitude B of the magnetic field vector or the magnetic flux density B thus becomes

$$B ( r , {t}_{j} ) = \sqrt{{B}_{x}^{2} ( r , {t}_{j} ) + {B}_{y}^{2} ( r , {t}_{j} ) + {B}_{x}^{2} ( r , {t}_{j} )}$$

The local field values may be used, for example, as an input for field calibration or field mapping (e.g., in the case of an offline field camera), or for correcting image artifacts and image distortion (e.g., for an inline field camera).

According to one embodiment, the magnetic field strength sensors are arranged such that the magnetic field strength sensors are arranged substantially on the surface of a notional sphere. The magnetic field strength sensors are, for example, arranged equidistantly on the surface of the notional sphere. Alternatively or additionally, the magnetic field strength sensors are arranged at least in part on a local coil. Placement on a spherical surface may be implemented, for example, similarly to the description in EP 4 407 334 A1. Such placement on spherical surfaces may correspond to an offline field calibration or offline field camera. Offline field cameras map the distribution of the magnetic field and use maps stored in this way to correct the data captured during a clinical scan. The implementation of the placement on a local coil may be provided, for example, similarly to the description in EP U.S. Pat. No. 11,815,575 B2. This implementation may correspond to an inline field camera.

The present embodiments also relate to a system for acquiring magnetic flux densities of a magnetic resonance device within a patient tunnel of the magnetic resonance device (e.g., of a gradient coil (18) within a patient tunnel (14) of the magnetic resonance device (1)). The system includes: a processing circuit; a storage medium; and a data interface. The storage medium includes a computer program with instructions that, on execution of the program, cause the processing circuit to carry out the method for acquiring magnetic flux densities of a gradient coil of a magnetic resonance device within a patient tunnel of the magnetic resonance device. The data interface is configured to receive the magnetic flux densities acquired by the at least one magnetic field strength sensor in the first spatial direction and in the second spatial direction. The above explanations regarding the disclosed method apply mutatis mutandis to the system.

In one example, the system includes a magnetic resonance device. The system may include at least one gradient coil and at least one magnetic field strength sensor (e.g., at least four magnetic field strength sensors or four “sensor arrays” that are arranged in different regions of the gradient coil and/or the system, for example, substantially in opposing regions of the gradient coil and/or substantially on a surface of a notional sphere). The at least one magnetic field strength sensor may optionally be part of the gradient coil and/or be fixedly connected to the gradient coil.

The system may include at least one patient couch that is configured to be moved into the patient tunnel of the magnetic resonance device. The patient couch includes at least one magnetic field strength sensor that is arranged on the patient couch such that the magnetic field strength sensor is arranged at a position within the patient tunnel at which the magnetic flux densities of the B0 magnetic field of the B0 main magnet of the magnetic resonance device are below the predefined value in the first spatial direction and/or in the second spatial direction. Such a magnetic field strength sensor may, for example, be arranged on a patient couch, such that the sensor may be moved into the patient tunnel together with the patient couch and may there be positioned as close as possible to the coordinate origin of X-Y-Z coordinate system.

The system may include at least one local coil that is configured to be arranged against or on a patient and to be moved into the patient tunnel of the magnetic resonance device. The local coil includes at least one magnetic field strength sensor (e.g., at least four magnetic field strength sensors) that is arranged on the local coil such that the at least one magnetic field strength sensor is arranged at a position within the patient tunnel at which the magnetic flux densities of the B0 magnetic field of the B0 main magnet of the magnetic resonance device are below the predefined value in the first spatial direction and/or in the second spatial direction. Such a local coil may, for example, be a head coil or a chest coil.

In order to disrupt a measurement of the magnetic resonance device as little as possible, in each case, a microcontroller may be provided adjacent to one or to a plurality of adjacent magnetic field strength sensors, and a sensor-controller unit may consequently be provided. This sensor-controller unit may be equipped with radio-frequency shielding (e.g., made of carbon-copper braid). Such a sensor-controller unit may then transmit measurement data to a computer unit by way of optical waveguides.

The present embodiments also relate to a computer program element with instructions that, on execution on data processing devices of a data processing environment, are set up to carry out the acts of the above-stated method in an above-stated system.

The present embodiments also relate to the use of at least one magnetic flux density acquired according to a method as described above as an input value for: a nominal/actual value comparison of magnetic flux densities; a method for image reconstruction; and/or monitoring at least one magnetic flux density.

As explained, the acquired magnetic flux densities may be used for various applications. For example, the gradient fields or change in gradient fields may be continuously acquired and monitored. The acquired magnetic flux densities may be used in the course of image reconstruction (e.g., in the event of undesired eddy current effects). It is also possible to make use of the magnetic flux densities for an emergency shutdown should magnetic flux densities falling outside a predefined range be acquired. It is consequently also possible to carry out a nominal/actual comparison of the magnetic flux densities, for example, in order to be able to carry out quality and/or operational evaluations with regard to the gradient coil.

### BRIEF DESCRIPTION OF THE DRAWINGS

All the embodiments described herein may be combined with one another, unless explicitly stated otherwise. Further features, advantages, and possible applications of the present invention are revealed by the following description, the example embodiments, and the figures, in which:

FIG. 1 shows a conventional magnetic resonance device;

FIG. 2 shows a schematic representation of a system according to an embodiment;

FIG. 3 shows a schematic representation of a method according to an embodiment;

FIG. 4 shows a diagram with X and Y field strength components of a B0 magnetic field;

FIG. 5 shows a first view of a gradient coil with a plurality of magnetic field strength sensors;

FIG. 6 shows a second view of the gradient coil from FIG. 5;

FIG. 7 shows a schematic view of an arrangement of a plurality of magnetic field strength sensors on a spherical surface that surrounds a defined volume;

FIG. 8 shows a schematic view of an arrangement of magnetic field strength sensors on a head coil; and

FIG. 9 shows a schematic representation of a method according to according to a further embodiment.

### DETAILED DESCRIPTION

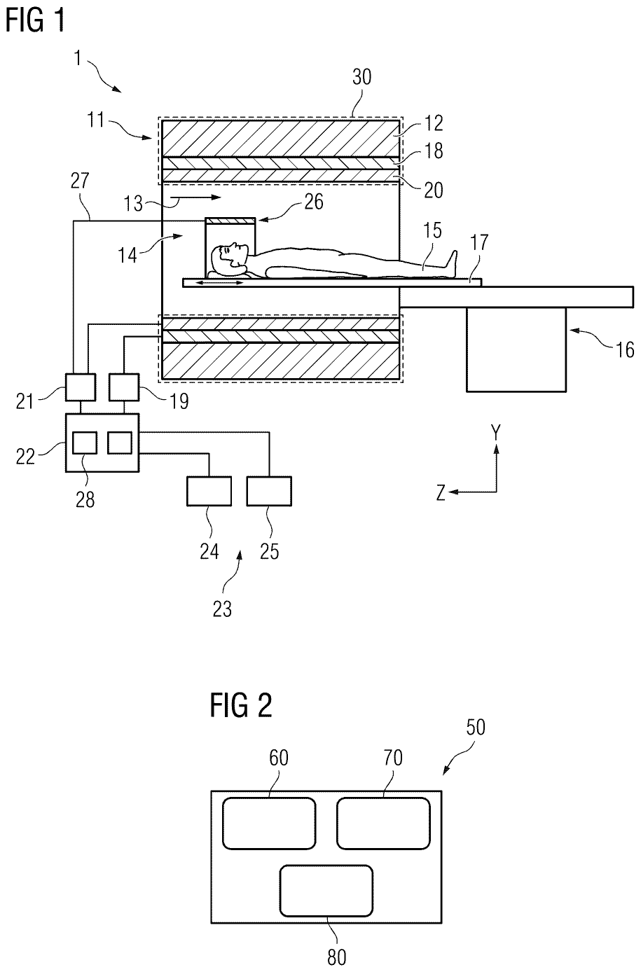

FIG. 1 depicts a conventional magnetic resonance device 1. The magnetic resonance device 1 includes a field generation unit 11 that has a B0 main magnet or B0 magnet 12 with one or more permanent magnets, electromagnets, or superconductive magnets for generating a strong and, for example, homogeneous B0 main magnetic field or B0 magnetic field 13. The magnetic resonance device 1 also includes a patient tunnel 14, also denoted bore 14, for accommodating a patient 15. The patient tunnel 14 is of cylindrical construction in the example embodiment shown and enclosed in a circumferential direction by the B0 main magnet 12. Configurations of the patient tunnel 14 that deviate from this example may, however, be provided. The patient tunnel 14 may substantially coincide with an image capture region of the magnetic resonance device 1.

In the example shown in FIG. 1, the patient 15 is positionable in the patient tunnel 14 using a patient positioning apparatus 16 of the magnetic resonance device 1. The patient positioning apparatus 16 has a horizontally movable patient table 17 for this purpose.

The field generation unit 11 has a gradient system with at least one gradient coil 18 for generating a magnetic gradient field that is used for spatial encoding during a magnetic resonance examination. The gradient coil 18 is controlled by a gradient control unit 19 of the magnetic resonance device 1. In one embodiment, the gradient system may include a plurality of gradient coils 18 for generating magnetic gradient fields along different spatial directions that may be oriented orthogonally to one another.

The field generation unit 11 also includes a radio-frequency system with a radio-frequency coil that in the present example embodiment is configured as a body coil 20 permanently integrated into the magnetic resonance device 1. The body coil 20 is configured to excite nuclear spins that are located in the main magnetic field 13 generated by the B0 main magnet 12. The body coil 20 is controlled by a radio-frequency control unit 21 of the magnetic resonance device 1 and emits radio-frequency excitation pulses into the image capture region, which is substantially formed by the patient accommodation zone 14 of the magnetic resonance device 1. The body coil 20 may also be configured to receive magnetic resonance signals and form a receive unit or part of a receive unit of the magnetic resonance device 1.

The magnetic resonance device 1 includes a control unit 22 for controlling the magnetic resonance device 1 (e.g., the gradient control unit 19 and the radio-frequency control unit 21). The control unit 22 is, for example, configured to coordinate the performance of an imaging sequence, such as, for example, a gradient echo (GRE) sequence, a turbo spin echo (TSE) sequence, or an ultra-short echo time (UTE) sequence. The control unit 22 also includes a computing unit 28 for evaluating magnetic resonance signals that are acquired with an imaging sequence during a magnetic resonance examination.

The magnetic resonance device 1 may include a user interface 23 that has a signal connection to the control unit 22. Control information, such as, for example, imaging parameters of the magnetic resonance examination, may be displayed on a display unit 24 (e.g., on at least one monitor) of the user interface 23. The display unit 24 may, for example, be configured to provide a graphical user interface with the depiction of a relevant body region of the patient 15. The user interface 23 also has an input unit 25 via which magnetic resonance measurement parameters may be input or modified by a user.

The magnetic resonance device 1 may have further components, such as, for example, a local coil 26. The local coil 26 may be positioned in a position appropriate to the application on a diagnostically or therapeutically relevant region of the body of the patient 15. The local coil 26 may have a plurality of antenna elements that are configured to acquire magnetic resonance signals from the relevant region of the body of the patient 15 and transmit the magnetic resonance signals to the computing unit 28 and/or the control unit 22. The local coil 26 may be connected to the radio-frequency control unit 21 and the control unit 22 via an electrical connection lead 27 or another signal connection. Similarly to the body coil 20, the local coil 26 may also be configured to excite nuclear spins in the jaw region 31 of the patient 15. The local coil 26 may be controlled by the radio-frequency control unit 21 for this purpose.

The field generation unit 11 and a magnet retaining structure are conventionally enclosed by a housing 30. The housing 30 may be configured to protect components of the magnetic resonance device 1 from external influences and/or to provide a touch guard for a patient 15.

FIG. 2 shows an example embodiment of a system according to the present embodiments 50 for acquiring magnetic flux densities of a gradient coil 18 of a magnetic resonance device 1 within a patient tunnel 14 of the magnetic resonance device 1, including: a processing circuit 60; a storage medium 70; and a data interface 80. The storage medium 70 includes a computer program with instructions that, on execution of the program, cause the processing circuit 60 to carry out the method for acquiring magnetic flux densities of a magnetic resonance device (e.g., of a gradient coil of a magnetic resonance device) within a patient tunnel of the magnetic resonance device. The data interface 80 is configured to receive the magnetic flux densities acquired by the at least one magnetic field strength sensor in the first spatial direction and/or in the second spatial direction.

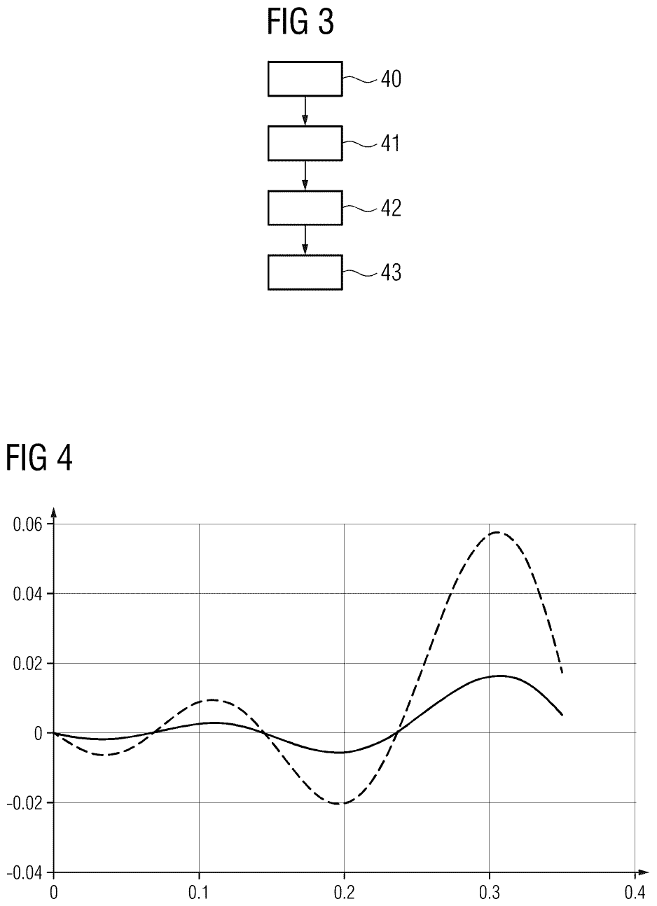

FIG. 3 is a schematic representation of a method for acquiring magnetic flux densities of a gradient coil 18 of a magnetic resonance device 1 within a patient tunnel 14 of the magnetic resonance device 1.

In a first act 40, at least one position within the patient tunnel 14 is ascertained at which the magnetic flux densities of the B0 magnetic field 13 of the B0 main magnet 12 of the magnetic resonance device 1 are below a predefined value in a first spatial direction (e.g., in the X direction) and/or in a second spatial direction (e.g., in the Y direction). The predefined value may amount substantially to 0.

FIG. 4 shows a diagram with X field strength components (represented by the dashed line) and Y field strength components (represented by the continuous line) (B0.x; B0.y) of a B0 magnetic field in a Z position. The values are shown in T. As is apparent from FIG. 4, there are a plurality of positions at which both the X field strength component (B0.x) and the Y field strength component (B0.y) are substantially zero. In other words, the magnetic flux density in these two spatial directions is substantially 0 there. These are the positions at which the magnetic field strength sensors may be arranged (e.g., oriented collinearly to the X field strength component (B0.x) and Y field strength component (B0.y)).

By way of example, it was possible to ascertain for a 60 cm B0 main magnet 12 the following coordinates/positions at which both the X field strength component (B0.x) and the Y field strength component (B0.y) are substantially zero:

$$Z = 0 cm ; Y = + / - 30 cm ; X = 0 cm ;$$

$$Z = + / - 7.6 cm ; Y = + / - 30 cm ; X = 0 cm ;$$

$$Z = + / - 7.6 cm ; Y = 0 cm ; X = + / - 30 cm ;$$

$$Z = + / - 7.6 cm ; Y = + / - 29 cm ; X = 7.6 cm ;$$

In a further act 41, a magnetic field strength sensor or, for example, a plurality of magnetic field strength sensors are arranged at one or more positions ascertained in the preceding act 40. The magnetic field strength sensors are arranged and configured to acquire the magnetic flux density at least in the first spatial direction and/or in the second spatial direction.

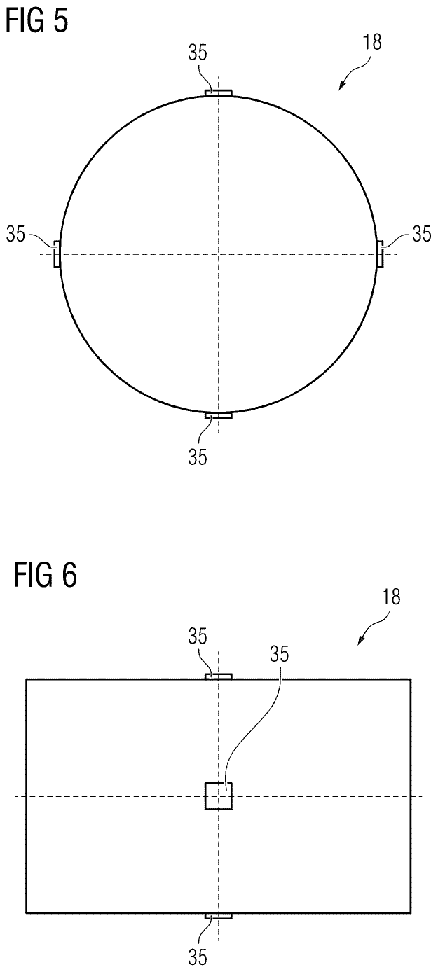

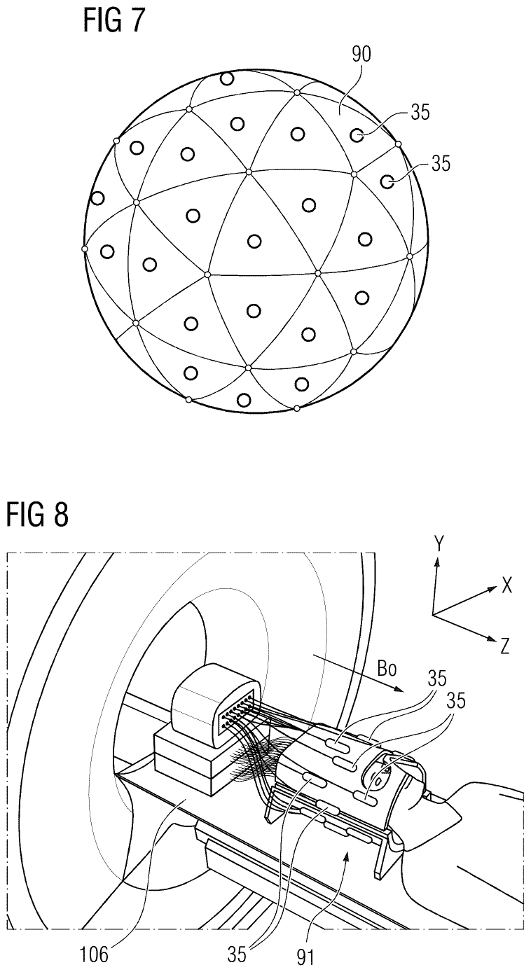

FIGS. 5 and 6 show a schematic view of the gradient coil 18 on which the plurality of magnetic field strength sensors 35 or sensor arrays 35 are arranged at positions that were ascertained in act 40. FIG. 7 is a schematic view of an arrangement of a plurality of magnetic field strength sensors 35 on a spherical surface 90 that surrounds a defined volume. FIG. 8, in contrast, shows a schematic view of an arrangement of magnetic field strength sensors 35 on a local coil (e.g., a head coil 91).

In act 42, the magnetic flux densities or their changes may then be acquired by the magnetic field strength sensors 35 in the first spatial direction (e.g., the X field strength component) and/or in the second spatial direction (e.g., the Y field strength component).

In a further, optional, act 43, a magnetic flux density may be calculated in a third spatial direction (e.g., the Z field strength component) based on the two acquired magnetic flux densities. In the configuration, the X and Y field strength components are acquired as described above. As explained, it is consequently possible to calculate or derive, for example, the Z field strength component from the “source freedom” of the magnetic flux density since there are only closed flux lines in the magnetic field. Applying Maxwell's second equation, which is also known as “Gauss' law for magnetic fields”, gives rise to the following:

$$∇ \cdot B = 0$$

which states that the magnetic field is source-free. The divergence of the vector field is the scalar field:

$$B = ( {B}_{x} , {B}_{y} , {B}_{z} )$$

is defined as

$$\frac{\partial {B}_{x}}{\partial x} + \frac{\partial {B}_{y}}{\partial y} + \frac{\partial {B}_{z}}{\partial z} = 0 ,$$

$$\frac{\partial {B}_{z}}{\partial z} ,$$

gives rise to:

and, solved according to

$$- ( \frac{\partial {B}_{x}}{\partial x} + \frac{\partial {B}_{y}}{\partial y} ) = \frac{\partial {B}_{z}}{\partial z}$$

Placing a plurality of magnetic field strength sensors therefore makes it possible not only to measure the X and Y field strength components of the magnetic flux densities, for example, locally, but also to determine the corresponding derivatives in both spatial directions (e.g., in the X and Y directions) in order to be able to ascertain the magnetic flux density in the Z direction.

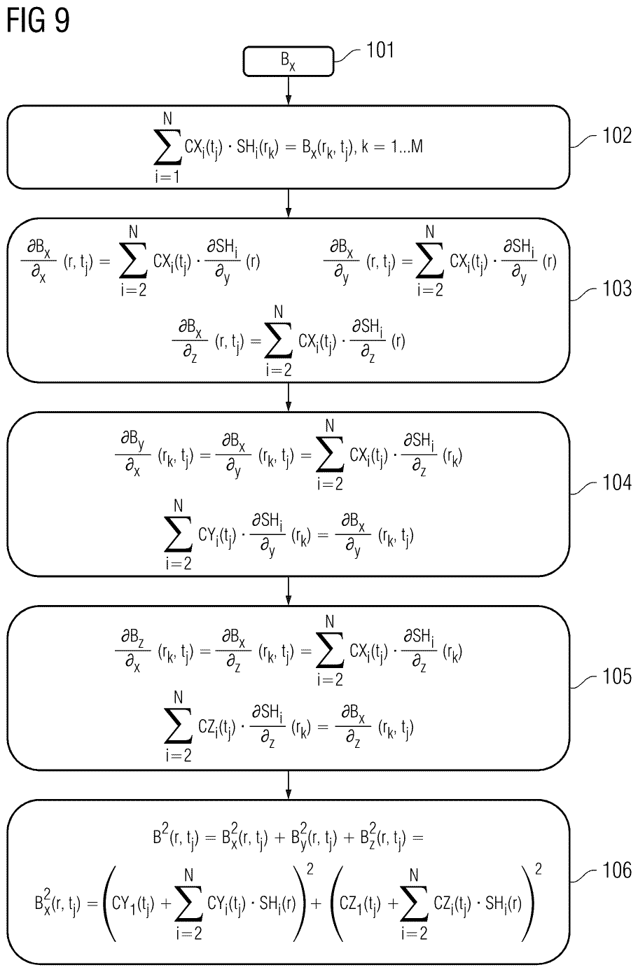

FIG. 9 shows a schematic representation of a method for acquiring magnetic flux densities of a magnetic resonance device 1 within a patient tunnel 14 of the magnetic resonance device 1. In a first act 101, a first spatial direction is ascertained in which the magnetic flux density of a B0 magnetic field 13 of a B0 main magnet 12 of the magnetic resonance device 1 within the patient tunnel 14 is below a predefined value at at least one position. This may, for example, be the X direction. A plurality of magnetic field strength sensors 35 are then arranged at a plurality of positions (e.g., at least four different positions) within the patient tunnel 14, and the magnetic flux densities 35 are acquired by the magnetic field strength sensors in the first spatial direction according to one Cartesian component (e.g., Bₓ). The magnetic field strength sensors may, for example, be arranged about a defined volume or on the surface of the volume (e.g., on the surface of a sphere as shown in FIG. 7 or on the surface of a local coil as shown in FIG. 8). In a further act 102, a first complete field model is fitted to the Cartesian component of the flux density in the first spatial direction by using regular spatial harmonic functions. In a further act 103, a first field gradient field model of the vectorial magnetic flux density for the Cartesian component of the first spatial direction is calculated based on the first complete field model. In a further act 104, a second field gradient model for the Cartesian component of a second spatial direction (e.g., B_{y}) that is orthogonal to the first spatial direction is calculated based on the first field gradient model. In a further act 105, a third field gradient model for the Cartesian component in a third spatial direction that is orthogonal to the first spatial direction and the second spatial direction is calculated based on the first field gradient model or the second field gradient model. For example, the third spatial direction may be oriented along the axis of symmetry of the main magnet (e.g., Z direction). In a further act 106, based on measurement data from two field probes from the patient tunnel (e.g., NMR field probes), an absolute value of the magnetic flux density is in each case used to determine the vectorial magnetic flux density at least at the two locations of the field probes and thus determine first-order model coefficients for the second complete field model and the third complete field model. Using the three field models, the magnetic flux density may be calculated at any desired location, at least within the defined volume. The acts of the method may be repeated for different (e.g., equidistant) points in time in order also to determine a time-dependent magnetic flux density.

The present invention is not limited to the embodiments described above. The terms “comprising” and “having” do not exclude any other elements or steps, and the indefinite article “a” or “an” does not exclude a plurality. Features or steps that have been described with reference to the above example embodiments may also be used in combination with other features.

Independent of the grammatical term usage, individuals with male, female, or other gender identities are included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for acquiring magnetic flux densities of a magnetic resonance device within a patient tunnel of the magnetic resonance device, the method comprising:
ascertaining a first spatial direction, a second spatial direction, or the first spatial direction and the second spatial direction in which the magnetic flux density/densities of a B0 magnetic field of a B0 main magnet of the magnetic resonance device within the patient tunnel is or are below a predefined value at at least one position;
arranging at least one magnetic field strength sensor at the at least one position within the patient tunnel, wherein the magnetic field strength sensor is arranged and configured to acquire the magnetic flux density at least in the first spatial direction, in the second spatial direction, or in the first spatial direction and in the second spatial direction; and
acquiring the magnetic flux density/densities using the at least one magnetic field strength sensor in the first spatial direction, in the second spatial direction, or in the first spatial direction and in the second spatial direction.

2. The method as claimed in claim 1, further comprising ascertaining the at least one position within the patient tunnel, such that, at the at least one position, the magnetic flux densities of the B0 magnetic field of the B0 main magnet of the magnetic resonance device are below the predefined value in the first spatial direction and in the second spatial direction,
wherein the at least one magnetic field strength sensor is arranged and set up such that axes of the magnetic field strength sensor are oriented collinearly to two spatial directions of the B0 magnetic field, the magnetic flux densities of which are acquired,
wherein the magnetic flux density is acquired using the at least one magnetic field strength sensor in the first spatial direction and in the second spatial direction, and
wherein the method further comprises ascertaining a magnetic flux density in a third spatial direction based on the acquired magnetic flux densities in the first spatial direction and in the second spatial direction.

3. The method of claim 1, wherein the at least one magnetic field strength sensor comprises a plurality of magnetic field strength sensors that are arranged at a plurality of positions within the patient tunnel,
wherein the at least one magnetic field strength sensor is configured to acquire the magnetic flux density in the first spatial direction,
wherein the magnetic flux densities are acquired in the first spatial direction by the plurality of magnetic field strength sensors,
wherein, based on the acquired magnetic flux densities in the first spatial direction, the magnetic flux densities, local derivatives of the magnetic flux densities, or the magnetic flux densities and the local derivatives of the magnetic flux densities in the second spatial direction, a third spatial direction, or the second spatial direction and the third spatial direction are ascertained, and
wherein a location-dependent magnetic flux density in a defined volume is ascertained in at least two spatial directions.

4. The method of claim 3, wherein the plurality of positions include at least four different positions.

5. The method of claim 3, wherein at least two field probes that are configured to acquire a local absolute value of a magnetic field strength are arranged in the patient tunnel,
wherein an absolute value of the magnetic field strength is in each case acquired by the at least two field probes,
wherein the magnetic flux densities are ascertained in the second spatial direction, in the third spatial direction, or in the second spatial direction and the third spatial direction both based on the acquired magnetic flux densities in the first spatial direction and based on the absolute values of magnetic field strength ascertained with the at least two field probes.

6. The method of claim 3, wherein the magnetic flux densities in the second spatial direction, in the third spatial direction, or in the second spatial direction and the third spatial direction are ascertained based on calculations using spatial harmonic functions.

7. The method of claim 1, wherein the at least one magnetic field strength sensor is arranged substantially on a surface of a notional sphere, and
wherein:
the at least one magnetic field strength sensor is arranged equidistantly on the surface of the notional sphere;
the at least one magnetic field strength sensor is arranged at least in part on a local coil; or
a combination thereof.

8. The method of claim 1, wherein the first spatial direction, the second spatial direction, or the first spatial direction and the second spatial direction are in each case oriented orthogonally to a third spatial direction that is parallel to an axis of symmetry of the B0 main magnet, and
wherein the first spatial direction and the second spatial direction are oriented orthogonally to one another.

9. The method of claim 1, wherein:
the predefined value of the magnetic flux densities is between −0.02 T and 0.02 T;
the at least one magnetic field strength sensor has a linear measurement range between 0.01 T and −0.01 T; or
a combination thereof.

10. The method of claim 1, wherein the at least one magnetic field strength sensor includes a Hall sensor, an anisotropic magnetoresistive sensor, a magnetic tunnel resistance sensor, or a fluxgate magnetometer.

11. A system for acquiring magnetic flux densities of a magnetic resonance device within a patient tunnel of the magnetic resonance device, the system comprising:
a processing circuit;
a storage medium; and
a data interface,
wherein the storage medium comprises a computer program with instructions that, on execution of the program, cause the processing circuit to acquire magnetic flux densities of a magnetic resonance device within a patient tunnel of the magnetic resonance device, the acquisition of the magnetic flux densities comprising:
ascertainment of a first spatial direction, a second spatial direction, or the first spatial direction and the second spatial direction in which the magnetic flux density/densities of a B0 magnetic field of a B0 main magnet of the magnetic resonance device within the patient tunnel is or are below a predefined value at at least one position;
arrangement of at least one magnetic field strength sensor at the at least one position within the patient tunnel, wherein the magnetic field strength sensor is arranged and configured to acquire the magnetic flux density at least in the first spatial direction, in the second spatial direction, or in the first spatial direction and in the second spatial direction; and
acquisition of the magnetic flux density/densities using the at least one magnetic field strength sensor in the first spatial direction, in the second spatial direction, or in the first spatial direction and in the second spatial direction, and
wherein the data interface is configured to receive the magnetic flux densities acquired by the at least one magnetic field strength sensor in the first spatial direction and in the second spatial direction.

12. The system of claim 11, further comprising at least one gradient coil and the at least one magnetic field strength sensor,
wherein the at least one magnetic field strength sensor comprises a plurality of magnetic field strength sensors arranged in different regions of the at least one gradient coil, the system, or the at least one gradient coil and the system.

13. The system of claim 12, wherein the plurality of magnetic field strength sensors are arranged in opposing regions of the gradient coil, substantially on a surface of a notional sphere, or in the opposing regions of the gradient coil and substantially on the surface of the notional sphere.

14. The system of claim 11, further comprising at least one patient couch configured to be moved into the patient tunnel of the magnetic resonance device,
wherein the patient couch comprises at least one magnetic field strength sensor that is arranged on the patient couch such that the at least one magnetic field strength sensor is arranged at a position within the patient tunnel at which the magnetic flux densities of the B0 magnetic field of the B0 main magnet of the magnetic resonance device are below the predefined value in the first spatial direction, in the second spatial direction, or in the first spatial direction and the second spatial direction.

15. The system of claim 11, further comprising at least one local coil that is configured to be arranged against or on a patient and to be moved into the patient tunnel of the magnetic resonance device,
wherein the at least one local coil comprises at least four magnetic field strength sensors that are arranged on the at least one local coil such that the at least four magnetic field strength sensors are arranged at a position within the patient tunnel at which the magnetic flux densities of the B0 magnetic field of the B0 main magnet of the magnetic resonance device are below the predefined value in the first spatial direction, the second spatial direction, or the first spatial direction and the second spatial direction.

16. A method of using at least one magnetic flux density, the method comprising:
acquiring magnetic flux densities of a magnetic resonance device within a patient tunnel of the magnetic resonance device, the acquiring of the magnetic flux densities comprising:
ascertaining a first spatial direction, a second spatial direction, or the first spatial direction and the second spatial direction in which the magnetic flux density/densities of a B0 magnetic field of a B0 main magnet of the magnetic resonance device within the patient tunnel is or are below a predefined value at at least one position;
arranging at least one magnetic field strength sensor at the at least one position within the patient tunnel, wherein the at least one magnetic field strength sensor is arranged and configured to acquire the magnetic flux density at least in the first spatial direction, in the second spatial direction, or in the first spatial direction and in the second spatial direction; and
acquiring the magnetic flux density/densities using the at least one magnetic field strength sensor in the first spatial direction, in the second spatial direction, or in the first spatial direction and in the second spatial direction; and
using the at least one magnetic flux density for:
a nominal/actual value comparison of magnetic flux densities;
a method for image reconstruction;
monitoring one or more magnetic flux densities of the at least one magnetic flux density; or
any combination thereof.
